(19) United States
(12) Patent Application Publication
(10) Pub. No.: US 2012/0083026 A1
(43) Pub. Date: Apr. 5, 2012

(54) Method for removing CO2 from a smoke or exhaust gas of a combustion process

(76) Inventors: Donat-Peter Häder, Mohrendorf (DE); Manfred Rührig, Lauf a.d. Pegnitz (DE)

(21) Appl. No.: 13/377,176

(22) PCT Filed: Jun. 28, 2010
(86) PCT No.: PCT/EP2010/059122

§ 371 Date: Dec. 9, 2011

(30) Foreign Application Priority Data
Jun. 26, 2009 (DE) 102009030712.5

(51) Int. Cl. A62D 3/02; C12M 1/00

(52) U.S. Cl. 435/266; 435/289.1; 435/262.5

(57) ABSTRACT

A method and arrangement for removing CO₂ from smoke or exhaust gas of a combustion process in a power plant are provided. The exhaust gases comprising CO₂ are fed into a tank in which cellular organisms, such as micro algae, are present, converting the CO₂ into biomass when nutrients are added. The micro algae and/or the generated biomass have magnetic particles added, which combines with the algae and/or the biomass. The biomass with the magnetic particles is separated in a magnetic separation stage or magnetic drum separator

### CROSS REFERENCE TO RELATED APPLICATIONS

This application is the US National Stage of International Application No. PCT/EP2010/059122 filed Jun. 28, 2010, and claims the benefit thereof. The International Application claims the benefits of German Patent Application No. 10 2009 030 712.5 DE filed Jun. 26, 2009. All of the applications are incorporated by reference herein in their entirety.

### FIELD OF INVENTION

The claimed invention relates to a method and an arrangement for removing CO₂ from a smoke or exhaust gas of a combustion process.

### BACKGROUND OF INVENTION

It has been recognized in the scientific community since the 1990s at the latest that there is a statistically significant change in the climate and that it is one of the causes of the rise in the concentration of carbon dioxide, abbreviated as CO₂, in the atmosphere. This suspicion, initially still bound up with major uncertainties, has constantly been strengthened in the course of research and following intense controversy about global warming, and nowadays is largely the scientific consensus. In the opinion of the overwhelming majority of scientists, the observed temperature data cannot be explained without taking account of the greenhouse gases in the atmosphere. It is intended that the consequences of global warming be reduced by means of climate protection.

The major part of the radiation reaching the earth from the sun can pass through the earth's atmosphere more or less unchecked. A large part of the radiation reflected by the earth on the other hand, in particular the proportion in the infra-red range of the spectrum, is absorbed by the CO₂ present in the atmosphere. This has the consequence that the atmosphere heats up. This property makes carbon dioxide into a so-called greenhouse gas. After water vapor, carbon dioxide is the most potent of the greenhouse gases in relation to its proportional quantity, even if the specific potencies of methane and ozone are higher. All the greenhouse gases together increase the average temperature on the earth's surface from approx −18 C to approx +15 C (natural greenhouse effect). Carbon dioxide accounts for a proportion of approx. 9% to 26% of this overall effect and is therefore jointly responsible to a considerable degree for the life-friendly climate of the earth.

The proportion of CO₂ in the earth's atmosphere was subject to considerable fluctuations in the course of the earth's history, which have various biological, chemical, and physical causes. For at least 650,000 years, however, the proportion has always been below 280 ppm. The CO₂ concentration in the last 10,000 years remained relatively constant at 280 ppm. The balance of the carbon dioxide cycle was therefore largely at break-even in this period. With the start of industrialization in the 19^{th} century, the proportion of CO₂ in the atmosphere rose to the present 381 ppm (in 2006) and is currently continuing to rise by an average of 1.5 ppm to 2 ppm per year.

Anthropogenic CO₂ emissions, that is to say those caused by man, are, due to global deforestation, only absorbed to a level of about 45% by the natural carbon dioxide sinks, for example by the phytoplankton inhabiting the world's oceans. As a result, carbon dioxide is accumulating in the atmosphere.

Due to the global warming and the presumed connections with the CO₂ concentration in the earth's atmosphere, the cause of which lies in the emission of greenhouse gases triggered by man, opportunities have been and are being sought to reduce the accumulation of CO₂ in the earth's atmosphere. One option is summarized under the keyword CO₂ sequestration. What is understood by CO₂ sequestration in this respect is the depositing of carbon dioxide that has been created in power plants for example. Sequestration is a part of the so-called CCS process (“Carbon Capture and Storage” for the purpose of low-CO₂ use of fossil raw materials during power generation. Here, CO₂ is to be separated from the combustion products of fossil energy sources and subsequently stored to prevent it getting into the earth's atmosphere.

‘Sequestration’ in the true sense is the designation for the storage of CO₂. The separation from the combustion products in the power plant process can be effected with various processes, for example following coal gasification (reduced-CO₂ IGCC power plant), combustion in an oxygen atmosphere or CO₂ scrubbing from the flue gas or exhaust gas of the power plant. Potential storage sites for the separated CO₂ are thought to comprise, in the first place, geological formations such as mineral oil reservoirs, natural gas reservoirs, saline ground water-bearing strata (so-called “aquifers”) and coal seams. Deep-sea storage is also being investigated, but is not appropriate due to the acidification of the world's oceans.

Research and projects to date were only concerned as a rule with the storage of liquid or gaseous CO₂ or in the form of dry ice. Alongside this, however, there is also the possibility of binding the CO₂ as biomass and storing it as carbon obtained from same or subjecting it to further processing in some other way. For example, biomass for energy use can be generated from the exhaust gas from a power plant process with the aid of microalgae when CO₂ is fed in.

One possibility for the implementation of CO₂ sequestration of this type consists in the exploitation of a process that takes place in nature. Single-celled organisms occurring in the world's oceans such as algae or cyanobacteria or phytoplankton respectively are responsible for around half of global carbon fixing by means of photosynthesis. The major part of this fixed carbon is returned to the atmosphere again in the form of CO₂ via the marine food chains. However, a small part of the biogenic carbon sinks to lower oceanic levels and as a result is extracted from the atmosphere for a long period. The latter process is strongly dependent on the iron available in the sea. However, parts of the world's seas are characterized by a lack of available iron. To promote the process and as a result, the fixing of carbon, fertilization of the seas with iron would consequently be an option. However, studies have shown that an algal bloom can be triggered by the addition of iron in these regions. An algal fertilization could mean that to some extent serious consequences arise for the marine ecosystems, which have not yet been adequately researched at the present time.

Alternatively, binding the CO₂ liberated during the combustion of fossil energy sources in biomass in the form of microorganisms, which, in conjunction with solar energy and further nutrients such as phosphate or nitrogen, are capable of fixing CO₂ in biomass by means of direct photosynthesis, is a known process. In this form of CO₂ sequestration, the combustion products or the flue gas respectively of fossil energy sources are directed, following corresponding cleaning (mainly of sulfur compounds), through a solution in which the organisms are present. The organisms can multiply exponentially in certain life stages, which has the consequence of a rapid build-up of biomass that to some extent lies markedly above those agriculturally grown plants such as elephant grass, sugar cane, and oil plant.

The breeding of the photosynthetically active cells or the organisms respectively occurs either in open systems, such as shallow ponds, or in bioreactors. Whereas the open systems are susceptible to the introduction of contamination from the air, which can lastingly harm or destroy the cell cultures respectively, the processes in bioreactors are more readily controllable. Due to the possibility of vertical construction, they potentially have a smaller space requirement, although they also necessitate a higher investment cost. The critical parameter for the efficiency of a system of this type is the biomass per system area and unit of time provided by the process.

Since the growth of cellular organisms such as algae follows so-called logarithmic growth laws, it is desirable, for the greatest possible cell growth rates, to set the population dynamics in the so-called log phase, that is to say the cells multiply exponentially and cells removed from the process can be reproduced again as quickly as possible. The precondition for obtaining exponential growth is the constant removal of cells from the process and the constant renewal of the basis of life for the cells, that is to say the renewal of the nutrients and also of the CO₂. To prevent unproductive start-up phases and saturation effects of the cells, the process should run as continuously as possible. Furthermore, a stable balance should establish itself between the quantity of cells growing back and the quantity of cells removed. Uncontrolled growth should consequently also be prevented since this means that a large part of the sunlight is absorbed in the cells near the surface and can no longer penetrate into deeper layers. Any interruption of the process and any renewed startup necessarily lead to production losses.

Removal processes are known from the state of the art. For example, suspensions are separated with the aid of centrifuges or decanters. However, as a rule these have a high energy requirement and consequently appear uneconomic for the purpose of separating cells. A further common process comprises microfiltration methods. A critical aspect in the case of these processes, however, in connection with the separation of what are, as a rule, very small cells that have a diameter of only few 10 μm, is blockage of the filters precisely in connection with algae, due to so-called biofouling. In the case of this process, which frequently runs in contact with water that is not germ-free, a slimy coating arises that very quickly clogs the microfilters used. However, frequent changing of filters has a strongly negative effect on the cost-effectiveness of processes of this type. Additionally, filters have to be laboriously backwashed for the purpose of obtaining the cells.

Apart from these physical/mechanical methods, chemical methods for removal are also known from the art. For example, in the case of so-called flotation methods, algae are bound by using injected gases and the addition of mostly foam-forming flotation agents, suspended, and skimmed off with the foam. Additionally, flocculation processes are known in which, for example, the solubility product of additives is exceeded by changing the pH value. The algae present in the suspension are also incorporated in the precipitated flakes, which algae can then be removed together with the flakes as sediment.

A disadvantage in the case of the chemical removal processes is, on the one hand, the addition of chemicals. Thus, in the case of the admixture of lye, a neutralization operation then has to be carried out, if it is wished to feed the alkaline process medium back into circulation. Flotation agents can often only be removed with difficulty and to some extent can have harmful effects on the biology of algal growth. Furthermore, the biomass separated in this way always still contains residues of the additive, which can often only be removed with difficulty.

### SUMMARY OF INVENTION

It is consequently an object of the present invention to disclose an alternative process with which carbon dioxide can be removed from a flue gas or exhaust gas stream from a combustion process.

This object is achieved by a method and an arrangement as claimed in the independent claims. Advantageous versions arise from the dependent claims.

According to the claimed invention, it is proposed, for removing CO₂ from the flue gas or exhaust gas of a combustion process, to bring at least part of the flue gas or exhaust gas into contact with organisms, in particular with cellular organisms, whereupon the organisms process at least part of the CO₂ contained in the flue gas or exhaust gas for the purpose of generating biomass. In this respect, magnetic particles are mixed with the organisms and/or the biomass generated. At least part of the biomass generated in this way is lastly separated in a magnetic separation stage.

By way of advantage in a first version, the flue gas or exhaust gas is brought into contact with the organisms in a first tank, whereupon the biomass is generated. The magnetic particles are fed into the first tank, which particles combine with the biomass generated. Lastly, at least part of the biomass generated is separated with the magnetic separation stage.

In an alternative version, the flue gas or exhaust gas is initially brought into contact with the organisms in a first tank. At least part of the biomass generated in the first tank is then fed into a further tank. The magnetic particles are mixed with the biomass in the further tank. Lastly, at least part of the biomass mixed with the magnetic particles is fed to the magnetic separation stage and separated with same.

By way of advantage, the process is carried out in such a way, that the generation of the biomass is effected in a multi-stage process.

Similarly, the cellular organisms can be precipitated in the form of flakes by the addition of additives prior to the removal of the biomass, whereupon the magnetic particles are incorporated at least in part in the flakes.

By way of advantage, the quantity of biomass removed per unit of time can be controlled by way of the quantity of magnetic particles added.

Cellular organisms grow back in the first tank. During the removal of the biomass, only as much biomass is removed that a stable balance establishes itself between the quantity of cells growing back and the quantity of cells removed.

By way of advantage, the removal of the biomass is effected continuously.

The biomass separated by the magnetic separation stage is processed to biogas in a fermentation process step. By way of advantage, magnetic particles are extracted, in a further magnetic separation step, from a residue of the biomass left behind during the fermentation process step.

Water is extracted, in a first process step, from the biomass separated by the magnetic separation stage. Alternatively or in addition, the biomass separated by the magnetic separation stage is pressed, for the purpose of obtaining vegetable oils, in a second process step, whereupon press residues are fed to the fermentation process step. The dewatered biomass from the first process step is fed to the second process step or the fermentation process step.

An inventive arrangement for removing CO₂ from the flue gas or exhaust gas of a combustion process has an exhaust gas pipe, via which the flue gas or exhaust gas is fed into a tank in which organisms, in particular cellular organisms, are present. The said organisms process at least part of the CO₂ present in the flue gas or exhaust gas to biomass. At least part of the organisms and/or the biomass generated is furnished with magnetic particles. Furthermore, a magnetic separation stage is provided with the aid of which at least part of the biomass generated can be separated.

Further advantages, features and details of the invention arise from the exemplary embodiment described in the following and also on the basis of the drawings.

### BRIEF DESCRIPTION OF THE DRAWINGS

FIG. 1 shows a fossil-fuel power plant with downstream CO₂ sequestration, and

FIG. 2 shows a system and also process steps for removing CO₂ from the flue gas or exhaust gas of a combustion process.

### DETAILED DESCRIPTION OF INVENTION

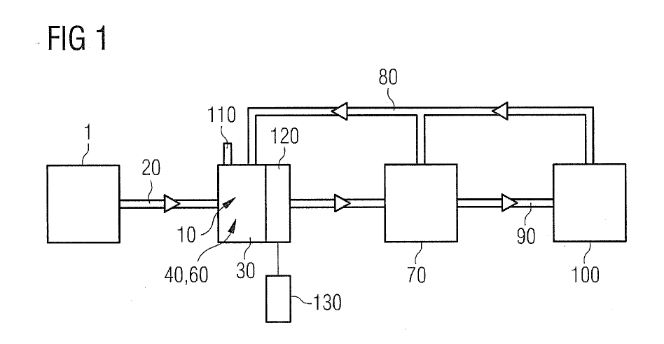

FIG. 1 shows, in a schematic representation, a power plant 1 with an exhaust gas pipe 20 via which the exhaust gas or flue gas respectively that arises during the combustion of a fossil energy source in the power plant 1 is routed away. In this respect, the exhaust gas of the combustion process in the power plant 1 contains harmful carbon dioxide, which is to be removed from the exhaust gas stream. The exhaust gas moves via the exhaust gas pipe 20 into a tank 30, which encloses a space in which cellular organisms 40 are present. The cellular organisms comprise for example photosynthetically active cells such as microalgae or bacteria, in particular cyanobacteria. The cellular organisms 40 convert the carbon dioxide existing in the exhaust gas stream of the power plant 1 into biomass 10, given the addition of nutrients such as phosphates or nitrogen, which enter the tank 30 or the space via an inlet aperture 110. This biomass 10 or at least part of it can be removed via a removal aperture 50 in the tank 30.

According to the invention, magnetic particles 60 are added to the cellular organisms 40, which particles consist in particular of magnetite. The cellular organisms 40 are capable, by way of their metabolism, of taking up not only the carbon dioxide from the exhaust gas stream and not only the nutrients but also the magnetic particles 60, and incorporating their cell structure or attaching themselves to the magnetic particles. To facilitate this step, functionalized magnetic particles 60 are employed in this version, which are enveloped in a protein or sugar coating for example, to increase the biological activity. By this means, the cellular organisms 40 that have taken up the magnetite 60 or attached themselves to same are given, for their part, a magnetic moment, and can subsequently be removed via a magnetic separation stage 120. As the magnetic separation stage 120, a magnetic drum separator or likewise other magnetic separators can be used for example. During this removal, the residual, magnetite-free cellular organisms 40 are not affected. Consequently, these continue to be available for the photosynthetic conversion of CO₂ into biomass. The rate of removal of cellular organisms 40 or of biomass can therefore be controlled by way of the quantity of magnetic particles 60 added.

The cellular organisms in the tank 30 constantly grow back in the case of the addition of nutrients. While taking account of the regrowth, the removal of the biomass 10 is controlled or regulated in such a way that only as much biomass 10 is removed that a stable balance establishes itself between the quantity of cells growing back and the quantity of cells removed. To this effect, a control and regulating facility 130 is provided, which controls or regulates the magnetic separation stage 120. The removal of the biomass 10 is ideally effected continuously.

The biomass (10) removed via the magnetic separation described is then subjected to further processing in a facility 70 designed in line with the desired use of the biomass 10. For example, a conversion of biomass into a primary energy source such as biogas, bioethanol or biodiesel can be effected in the facility 70. Depending on the oil content of the cellular organisms 40 employed, the biomass 10 can be pressed direct in the facility 70 for the purpose of obtaining vegetable oils. The press residues, which possibly still possess a high proportion of magnetic particles 60, can be fed back direct into the tank 30 via a pipe 80 to compensate for the loss of removed magnetic particles 60. The pressed oil can be removed via a removal aperture 90 in the facility 70 and be piped to a further magnetic separator 100 to recover the magnetite residues contained in the oil also. The said residues can also be fed into the tank 30 again via the pipe 80.

If the biomass 10 removed at the removal aperture 50 from the tank 30 is processed in some other way, for example fermented to methanol or to methane, aqueous suspensions are employed as a rule in this respect. The magnetic particles can likewise be recovered from same at a suitable point and be fed to the original sequestration process in the tank 30 again.

In an alternative version, a flocculation process takes place in the tank 30 for the purpose of removing the cellular biomass 10, whereupon magnetic particles such as magnetite particles are incorporated in the flakes arising in the process. The flakes formed are removed from the process stream via a magnetic separation process as already described above.

In the case of this method for removing the biomass while utilizing the flocculation process, the cellular organisms are precipitated in the form of flakes by the addition of additives in the presence of magnetic particles 60, in an interposed process. In this respect, the existing magnetic particles 60 are incorporated at least in part in the precipitated flakes, so that the said flakes can be removed via the magnetic separation process.

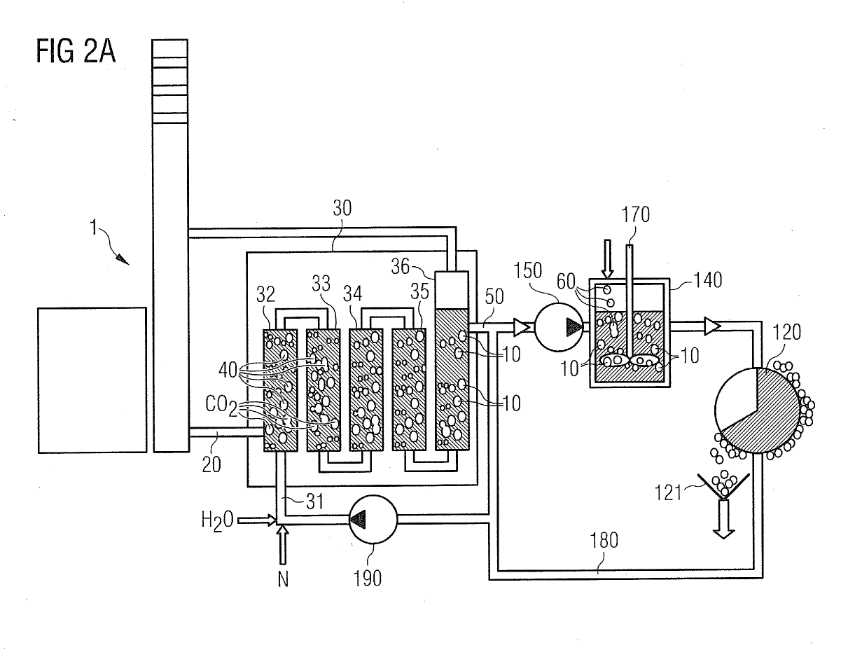

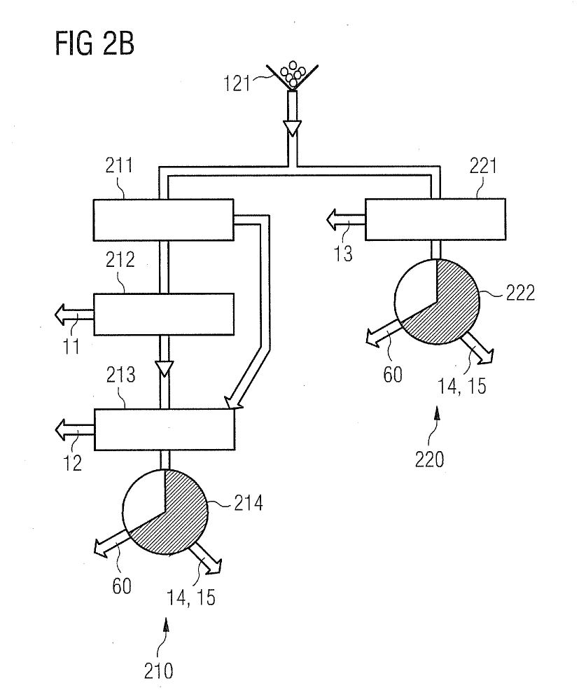

An alternative version of a system for CO₂ sequestration together with a flow diagram is represented in FIGS. 2A and 2B. As can be seen in FIG. 2A, at least part of the exhaust gas of the power plant 1 is removed via an exhaust gas pipe 20 of a power plant 1. The exhaust gas removed moves into a tank 30 and there it is led in a multistage process through spaces 32-36 in which cellular organisms 40 are present. Apart from the exhaust gas, nutrients N and also water and possibly, as described below, biomass are also fed into the tank 30 via an inlet 31.

As described above, the cellular organisms 40 convert the carbon dioxide existing in the exhaust gas stream into biomass 10. Lastly, the biomass generated is removed from the last space 36 in the series.

In contrast to the version in FIG. 1, a further tank 140 is provided here, in which magnetic particles 60 are fed into the biomass. In FIG. 1, this already occurred in the tank 30.

The biomass 10 removed from the space 36 is fed into the further tank 140 via a removal aperture 50 in the tank 30. A pump 150 serves the purpose of conveying the biomass 10. The magnetic particles 60 are fed into the tank 140 and mixed with the biomass 10 with the aid of a stirring facility 170 so that, as likewise described above, the object is achieved that the biomass 10 or the cellular organisms 40 take up the magnetic particles 60 or attach themselves to the magnetic particles 60. By this means, the cellular organisms 40 (and as a result the biomass 10) that have taken up the magnetite 60 or have attached themselves to same are for their part given a magnetic moment.

Following on from the further tank 140, a magnetic separation stage 120 is provided. This can be designed as a magnetic drum separator for example. The biomass 10 furnished with a magnetic moment is separated by the drum separator 120 and removed via an outlet 121 in the separator 120. The further processing of the biomass 10 removed here is described in connection with FIG. 2B. Biomass not separated by the separator 120 is fed via a corresponding pipe and into the further tank 140 again with the aid of the pump 150 and/or into the tank 30 with the aid of the pump 190.

The further processing of the separated biomass 10 is represented in a flow diagram in FIG. 2B. Two branches 210, 220 are provided for the purpose of processing: in the branch 210, water 11 is initially extracted from the separated biomass 10 in a first process step 211. In a second step 212, the dewatered biomass 10 is pressed, for example for the purpose of obtaining vegetable oils, whereupon the oil 12 obtained is removed. The press residues, which possibly still possess a high proportion of magnetic particles 60, are processed in a third process step 213, which comprises a fermentation process, to biogas 13, which is likewise removed. The magnetic particles 60 contained in the residues of the biomass 10 left behind in this respect are extracted from same in a fourth process step 214 or a further magnetic separation step 214 in a further magnetic separator, and for example fed into the further tank 140 again. Then just water 14 and also organic residues 15 are left behind. Alternatively, the biomass dewatered in the first process step 211 can also be fed direct to the fourth process step 214 by bypassing the second process step 212.

In the branch 220, the biomass 10 separated in the drum separator 120 is fed, in a first process step 221, to a fermentation process in which biogas 13 is generated. As in the branch 210, the magnetic particles 60 contained in the residues of the biomass 10 left behind in this respect are extracted from same in a second process step 222 or a further magnetic separation step 222 in a further magnetic separator, and for example fed into the further tank 140 again. Here also, water 14 and also organic residues 15 are left behind.

1.-13. (canceled)

14. A method for removing CO₂ from flue gas or exhaust gas of a combustion process, comprising:
exposing cellular organisms to at least part of the flue gas or exhaust gas,
processing at least part of the CO₂ contained in the flue gas or exhaust gas by the cellular organisms thereby generating biomass,
adding magnetic particles to the cellular organisms and/or generated biomass, and
separating at least part of the generated biomass via a magnetic separation stage.

15. The method as claimed in claim 14, further comprising:
providing a first tank,
wherein the cellular organisms are exposed to the at least part of flue gas or exhaust gas in the first tank, and
wherein the magnetic particles are fed into the first tank.

16. The method as claimed in claim 14, further comprising:
providing a first tank,
wherein at least part of the biomass is generated in the first tank and is fed into a second tank,
wherein the magnetic particles are added to the biomass in the second tank, and
wherein at least part of a mixture comprising the biomass and the magnetic particles is fed to the magnetic separation stage and separated via the magnetic separation stage.

17. The method as claimed in claim 14, wherein the generating of the biomass is effected in a multi-stage process.

18. The method as claimed in claim 14, wherein the magnetic particles are taken up by the organisms and/or the generated biomass and are incorporated in a cell structure of the organisms and/or the generated biomass.

19. The method as claimed in claim 14, wherein the magnetic particles attach themselves to the organisms and/or the generated biomass.

20. The method as claimed in claim 14, wherein the cellular organisms are precipitated in form of flakes by an addition of additives prior to a removal of the biomass, whereupon the magnetic particles are incorporated at least in part in the flakes.

21. The method as claimed in claim 14, wherein a quantity of the biomass removed per unit of time is controlled by way of a quantity of magnetic particles added.

22. The method as claimed in claim 15, wherein the cellular organisms grow back in the first tank, whereupon, during a removal of the biomass, only as much biomass is removed such that a stable balance establishes between a quantity of cells growing back and a quantity of cells removed.

23. The method as claimed in claim 14, wherein a removal of the biomass is effected continuously.

24. The method as claimed in claim 19, wherein the removal of the biomass is effected continuously.

25. The method as claimed in claim 21, wherein the removal of the biomass is effected continuously.

26. The method as claimed in claim 14, further comprising:
processing separated biomass, which has been separated by the magnetic separation stage, to biogas in a fermentation process step.

27. The method as claimed in claim 26, wherein the magnetic particles are extracted in a further magnetic separation step from a residue of the biomass left behind during the fermentation process step.

28. The method as claimed in claim 26, further comprising:
extracting water from the separated biomass in a first process step, and/or
pressing the separated biomass thereby obtaining vegetable oils in a second process step, wherein press residues are fed to the fermentation process step, and
wherein dewatered biomass from the first process step is fed to the second process step or the fermentation process step.

29. An arrangement for removing CO₂ from flue gas or exhaust gas of a combustion process, comprising:
a tank,
an exhaust gas pipe comprising cellular organisms,
wherein the flue gas or exhaust gas is fed into a tank via the exhaust gas pipe,
wherein the cellular organisms process at least part of CO₂ present in the flue gas or exhaust gas to biomass,
wherein at least part of the organisms and/or the biomass is furnished with magnetic particles; and
a magnetic separation stage for separating at least part of the biomass.
